(19) United States
(12) Patent Application Publication
(10) Pub. No.: US 2004/0199247 A1
(43) Pub. Date: Oct. 7, 2004

(54) Covering composition for drug-release stent and drug-release stent manufactured using same

(76) Inventors: Sung-Gwon Kang, Gwangju city (KR); Don Haeng Lee, Incheon-city (KR); Kyoo-Baek Lee, Seoul (KR)

Correspondence Address: BIRCH STEWART KOLASCH & BIRCH, PO BOX 747, FALLS CHURCH, VA, 22040-0747, US

(21) Appl. No.: 10/487,906

(22) PCT Filed: Aug. 28, 2002
(86) PCT No.: PCT/KR02/01621

(30) Foreign Application Priority Data
Aug. 29, 2001 (KR) 20010052406

(51) Int. Cl. A61F 2/06

(52) U.S. Cl. 623/1.42; 623/1.46

(57) ABSTRACT

Disclosed is a covering composition for a drug-release stent, and a drug-release stent manufactured by using the same. The covering composition includes polyurethane, polyethyleneglycol, a drug, and an organic solvent.

### CROSS REFERENCE TO RELATED APPLICATION

This application is based on application Ser. No. 2001-52406 filed in the Korean Industrial Property Office on Aug. 29, 2001, the content of which is incorporated hereinto by reference.

### BACKGROUND OF THE INVENTION

(a) Field of the Invention

The present invention relates to a covering composition for a drug-release stent and a drug-release stent manufactured using the same, and more particularly, to a covering composition for a drug-release stent which is capable of controlling a drug-release rate and is specially useful for introduction of large sized substances such as killed bacteria or polypeptides for immune reaction.

(b) Description of the Related Art

In surgical or other related invasive medicinal procedures, the insertion and expansion of stent devices in blood vessels, urinary tracts, or other difficult-to-access places for the purpose of preventing restenosis, providing support or reinforcement of vessel or lumen wall, and for other therapeutic or restorative functions, has become a common from of long-term treatment.

Recently, the general idea of utilizing implanted stents to carry medicinal agents, such as thrombolytic agents, or antiproliferative agent, has been developed. U.S. Pat. No. 5,092,877 discloses a stent of a polymeric material that may be employed with a coating associated with the delivery of drugs, and WO 96/32097 discloses a drug-releasing coated stent.

A method of producing a coated stent or a covered stent includes adding drugs to a solution including a polymer and coating the resulting mixture on a stent without or with filler such as a rod within the stent lumen, followed by drying. The resulting stent has a polymer layer with a biological active species.

However, the drug-release stent cannot suitably control the drug-release rate according to the type of drugs or patients' condition. In addition, stents for use in immune reaction therapy, which is the introduction of large-sized substances such as inactive bacteria or proteins for biological reaction reinforcement, have not been developed

### SUMMARY OF THE INVENTION

It is an object of the present invention to provide a covering composition for a drug-release stent, that is capable of controlling a drug-release rate.

It is another object to provide a covering composition for a drug-release stent that is useful for fostering an immune reaction through the injection of inactive bacteria or proteins into a patient.

It is still another object to provide a drug-release stent produced using the covering composition.

These and other objects may be achieved by a covering composition for a drug-release stent including polyurethane, polyethyleneglycol, a drug, and an organic solvent.

In order to achieve these objects and others, the present invention provides a drug-release stent including a tubular metal wire body having open ends and a thin open porous side wall structure, and a covering layer on an outer surface of the body. The covering layer includes a drug, polyethyleneglycol, and polyurethane.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings, wherein:

FIG. 1 is a graph showing drug-release results of drug-release stents according to Examples 1 to 3 of the present invention;

FIG. 2 is a schematic diagram of one embodiment of a stent according to the present invention; and

FIG. 3 is a schematic diagram of another embodiment of a stent according to the present invention.

### DETAILED DESCRIPTION OF THE INVENTION

The present invention relates to a covering composition for coating a drug-release stent. The composition includes polyurethane, polyethyleneglycol, and a drug. Polyurethane is nondegradable polymer in vivo, so it does not degrade in vivo. Polyethyleneglycol is soluble in water, and it is released in water or in vivo. Thus, when the stent of the present invention with a covering layer using the covering composition is inserted into a human body, polyethyleneglycol is released from the covering layer and polyurethane is not released from the covering layer, thereby making a porous side wall, matrix-type system.

A matrix-type system using techniques for controlling drug-release, such as those using a release controlling layer, ion-exchange, osmosis, and other systems, is suitable for controlling the release of macromolecules such as proteins.

A method of preparing the covering composition of the present invention will be illustrated below.

Polyethyleneglycol is dissolved in an organic solvent. Polyurethane with a molecular weight of 3000 to 10,000 is preferable to control the drug-release rate.

The organic solvent is any solvent as long as it dissolves polyethyleneplycol as well as polyurethane. Examples are tetrahydrofurane, dimethyl formamide, or dimethyl acetamide.

The amount of organic solvent is sufficient to readily dissolve polyethyleneglycol and to attain viscosity after the addition of polyurethane to coat the composition on the stent, and it is not limited.

The resulting solution is mixed with a drug. The drug may be an agent enhancing biological immunity (e.g. killed bacteria, proteins), or an anticancer agent (e.g. adriamycin, cisplatin, 5-FU).

Polyurethane is admixed to the resulting mixture to prepare a covering composition for a drug-release stent.

The mixing ratio of polyethyleneglycol and polyurethane is critical for release. If a stent manufactured by using a composition with an excessively high polyurethane content is inserted into a body, polyethyleneglycol may not be effectively released from the covering layer in the stent, which makes insufficient number of pores to the outer surface and blocks drug-release.

The amount of polyethyleneglycol is preferably equal to or less than 30 wt %, and more preferably 15 to 25 wt %; and that of polyurethane is preferably equal to or more than 70 wt %, and more preferably 85 to 75 wt %. If the amount of polyethylene glycol is more than 30 wt %, the strength of the covering layer decreases, thereby collapsing the covering layer.

The drug composition of the present invention may further include a pharmaceutical aqueous electrolyte.

A method of producing a stent using the covering composition will be described below.

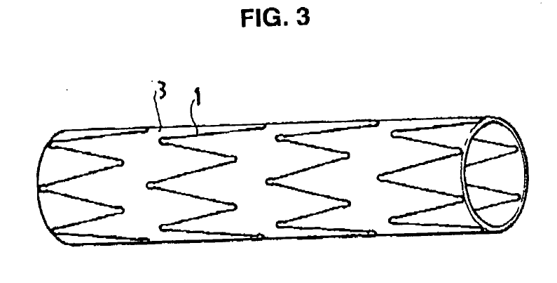

A stent body is produced using a metal wire having good elasticity and corrosion-resistance. The metal may be a shape-memory alloy, or stainless steel. The stent body can have various forms, and it generally has a tubular form having open ends and a thin open porous side wall structure. Examples are presented in FIGS. 2 and 3. Hereinafter, the structure of the stent is explained in below with reference to the accompanying FIG. 2.

The stent body includes a cylindrical tubular portion 1 and a movement-prevention portion 5. The cylindrical tubular portion can be designed to be any convenient diameter, and it is formed of a number of metal wires that are extended in a helix configuration and are axially displaced in relation to each other.

The movement-preventing portion 5 has a diameter that is larger than that of the tubular portion 1, and it is formed of a number of metal wires that extend in a zigzag configuration. Alternatively, the present invention can be applied to any form of stent, e.g. one without a movement-prevention portion or one with a tubular portion formed of metal wires that extend in a zigzag configuration.

FIG. 3 shows a tubular stent with the cylindrical tubular portion 1, without the movement-prevention portion.

The stent body is coated with the covering composition of the present invention.

The coating may be applied by dip-coating or spray-coating, or by any other technique to which the particular polymer/biological active agent combination is well suited. In addition, any pharmaceutically acceptable coating procedure may be applied to the coating process.

The resulting stent is dried to remove solvent from the covering composition. As a result, a polymer covering layer including polyurethane, polyethyleneglycol, and a drug is formed on a surface or on an outer surface or the stent. That is, the metal wires of the stent body are totally coated or covered with the polymer so that surfaces of the metal wires are coated or covered with the polymer layer including the biologically active materials. The obtained stent includes a tubular metal wire body having open ends and a thin open porous side wall structure and a covering layer on a surface or a outer surface of the stent.

When the resulting stent is inserted into a body, polyurethane is not dissolved in vivo but the polyethyleneglycol is dissolved and released. As a result, a polyurethane porous matrix is formed on the stent. The drug is diffused into the matrix, and is released from the stent to a human body.

The drug-release rate depends on the molecular weight of the polyethyleneglycol. Entanglement between the polyethyleneglycol and the polyurethane, which occurs due to the use of a higher molecular weight polyethyleneglycol, decreases the drug-release rate. A low molecular weight polyethylene does not incur the entanglement and does not decrease the drug-release rate, so the drug-release rate from the stent of the present invention can be controlled according to the type of the drug used and a patient's condition, based on the molecular weight of the polyethyleneglycol used.

The present invention is further explained in more detail with reference to the following examples, which further explain the scope of this invention.

### EXAMPLE 1

700 mg of tetrahydrofurane with an average molecular weight of 8000 was dissolved in 5.6 g of tetrahydrofurane. The resulting solution was mixed with OK432 5 vial (14 mg) as a drug. 4 g of polyurethane was added to the mixture, and it was completely dissolved with a magnetic stirrer to prepare a covering composition. The weight ratio of tetrahydrofurane:polyurethane was 20:80 (700 mg:4 g).

While the viscosity of the covering composition was measured with a viscometer, dimethylacetate was added to the covering composition to adjust it to a sufficient viscosity to coat on a stent.

The resulting composition was coated on the surface of a stent with a rotator, and it was dried in a 40° C. oven for 24 hours followed by vacuum-drying to completely remove the remaining organic solvent, and to produce a drug-release stent.

### EXAMPLE 2

A drug-release stent was produced by the same procedure as in Example 1, except that polyethyleneglycol with an average molecular weight of 3400 was used.

### EXAMPLE 3

A drug-release stent was produced by the same procedure as in Example 1, except that polyethyleneglycol with an average molecular weight of 10000 was used.

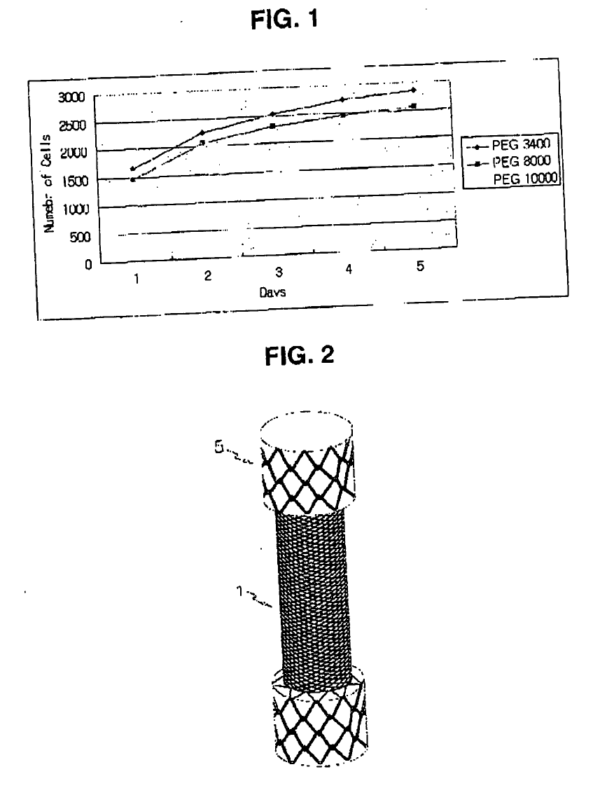

Drug-release tests on the covered stents according to Examples 1 to 3 were performed, and the results are presented in FIG. 1. In FIG. 1, PEG refers to polyethyleneglycol.

As shown in FIG. 1, the molecular weight of polyethylene glycol is inversely proportional to a slope. A lower molecular weight has a higher drug-release rate, and the higher molecular weight of 10,000 has a lower drug-release rate. It is expected from the results that the covering composition of the present invention can modify the drug-release rate from the stent.

While the present invention has been described in detail with reference to the preferred embodiments, those skilled in the art will appreciate that various modifications and substitutions can be made thereto without departing from the spirit and scope of the present invention as set forth in the appended claims.

What is claimed is:

1. A covering composition for a drug-release stent comprising:
polyurethane;
polyethyleneglycol;
a drug; and
an organic solvent.

2. The covering composition of claim 1, wherein the drug is selected from the group consisting of agents enhancing biological immunity and anticancer agents.

3. The covering composition of claim 1, wherein the amount of polyethyleneglycol in the covering composition is equal to or less than 30 wt %.

4. A drug-release stent comprising:
a tubular metal wire body having open ends and a thin open porous side wall structure; and
a covering layer on an outer surface of the tubular metal wire body, the covering layer comprising a drug, polyethyleneglycol, and polyurethane.

5. The drug-release stent of claim 4, wherein the drug is selected from the group consisting of agents enhancing biological immunity and anticancer agents.
